(19) United States
(12) Patent Application Publication
(10) Pub. No.: US 2014/0275741 A1
(43) Pub. Date: Sep. 18, 2014

(54) SYSTEM AND METHOD PROVIDING ALL-NIGHT SLEEP MANAGEMENT

(71) Applicant: HEADWATERS, INC., Marblehead, MA (US)

(72) Inventors: RUDY A. VANDENBELT, OTTAWA (CA); TROY G. ANDERSON, MARBLEHEAD, MA (US); PHILIPPE J. GENEREUX, OTTAWA (CA)

(73) Assignee: HEADWATERS, INC., MARBLEHEAD, MA (US)

(21) Appl. No.: 13/796,555

(22) Filed: Mar. 12, 2013

(51) Int. Cl. A61M 21/02

(52) U.S. Cl. CPC A61M 21/02
USPC 600/28; 600/26

(57) ABSTRACT

System and method of all-night sleep management integrates the sleep cycle into the wake cycle with the result that the individual's sense of wholeness and overall well-being is promoted and improved day-in-day-out. In different embodiments, the system and method of all-night sleep management provides audio that a media player plays without potentially disruptive transitions to help people to manage tinittus or help babies or others to fall and stay asleep or to prevent failing to fall asleep or to prevent waking up of otherwise sensitive sleepers and provides an audio, video or other signal that an audio, video or other media player plays incorporating pacing, binaural beats, waveform manipulation, brainwave entrainment, light frequency, amplitude and color modulation or other techniques to draw people from the awake state, through one or more initial, staged intermediate sleep stages and back into the awake state. The media may be prerecorded media or may be provided by a programmed controller in response to user input selection.

### FIELD OF THE INVENTION

This invention is drawn to the field of sleep therapy, and more particularly, to novel system and method providing all-night sleep management.

### BACKGROUND OF THE INVENTION

Sleep is an integral part of the whole sleep-wake cycle. Generally, the better the sleep phase is integrated into the whole cycle the more the awake phase expresses its natural vigor, acuity and wholesomeness. Conversely, the less the sleep phase is integrated into the sleep-wake cycle, the more tired, less mentally clear and irritable is the awake phase.

Techniques heretofore to promote rest and to improve sleep have typically sought to induce rest if not sleep by use of natural or other sounds intended to soothe and comfort, and/or to cancel out or to mask room noise. Discrete sound tracks prerecorded on albums, CDs, and MP3 files themed for relaxation and to mask noise are known. Digital sound machines also are known which play and repetitively replay soothing and/or sleep inducing sounds stored in internal and/or external memory, such as the IMPROVED FLEXIBILITY DIGITAL SOUND RELAXATION SYSTEM of U.S. Pat. No. 5,867,580, incorporated herein by reference, which induces rest and sleep by repetitive replay of audio content in looped and/or sound bite format, and the DIGITAL SOUND RELAXATION AND SLEEP-INDUCING SYSTEM AND METHOD of U.S. Pat. No. 7,749,155, incorporated herein by reference, which induces rest and sleep, among other things, by progressively slower replay of looped audio content.

However, the utility of the heretoknown techniques to promote rest and/or induce sleep has been limited by the soundtracks provided; on the one hand, the discrete soundtracks necessarily introduce gaps in the listener's acoustic field when a track is replayed, while the sound machine soundtracks in looped format introduce artifacts that, consciously and/or unconsciously, tend to disturb the rest, and possible sleep, of more sensitive sleepers.

Even the softest noise or noticeable pattern can be enough to wake a sleeping person or prevent a person from falling asleep. This may occur, for example, each time a so-called “seamless” loop sound repeats itself or each time a “white noise” sound sample is repetitively replayed. Each such event presents the opportunity to disturb sleep and/or disrupt rest and/or prevent sleep.

There is thus the need for system and method providing all-night sleep management that is not subject to the disadvantages of the heretofore known techniques to promote rest and/or induce sleep.

### SUMMARY OF THE INVENTION

Accordingly, one object of the present invention is to disclose system and method of all-night sleep management to better integrate the sleep cycle into the wake cycle with the result that the individual's sense of wholeness and overall well-being is promoted and improved day-in-day-out.

Another object is to disclose system and method of all-night sleep management that better integrates the sleep cycle into the wake cycle of average users and/or that is customized to each individual sleeper.

A further object is to disclose system and method of all-night sleep management providing audio that a media player plays without potentially disruptive transitions to help people to manage tinittus or help babies or others to fall and stay asleep or to prevent failing to fall asleep or to prevent waking up of otherwise sensitive sleepers.

A further object is to disclose system and method of all-night sleep management providing an audio, video or other signal that an audio, video or other media player plays incorporating pacing, binaural beats, waveform manipulation, brainwave entrainment, light frequency, amplitude and color modulation or other techniques to draw people from the awake state, through one or more staged sleep stages and back into the awake state.

In accord with these and other objects, the present invention discloses apparatus for use in an all-night sleep management system, comprising media for play on a media player that is part of said all-night sleep management system that when played on the media player has a duration that corresponds to the duration of the overall awake-to-sleep-to-wake-up-refreshed sleep cycle of a predetermined sleeper whose sleep cycle is being managed by the media player of the all-night sleep management system and has predetermined content stored thereon determined to implement all-night sleep management of the sleep cycle of the predetermined sleeper's sleep cycle being managed by the media player of the all-night sleep management system. In one embodiment, the media of duration that corresponds to the duration of the overall awake-to-sleep-to-wake-up-refreshed sleep cycle is audio media having prerecorded natural, tinittus or other noise masking, rest promoting or sleep inducing sounds stored thereon having a repeat cycle, wherein the repeat cycle is no less in duration than the duration of the overall awake-to-sleep-to-wake-up-refreshed sleep cycle of the predetermined sleeper's sleep cycle being managed by the media player of the all-night sleep management system. In other disclosed and exemplary embodiments, the media of duration that corresponds to the duration of the overall awake-to-sleep-to-wake-up-refreshed sleep cycle is audio and/or video media that includes a go-to-sleep segment having content adapted to facilitate falling asleep, a wake-up segment having content adapted to facilitate waking up and at least one segment intermediate said go-to-sleep segment and said wake-up segment having content adapted to manage intermediate sleep stages, said segments cooperating to provide a duration that corresponds to the duration of the overall awake-to-sleep-to-wake-up-refreshed sleep cycle of a predetermined sleeper whose sleep cycle is being managed by the media player of the all-night sleep management system.

In different disclosed embodiments, the media could be streamed media and the media player could be an audio player such as an MP3 or other audio player that is a stand alone player or a player running on a smart phone or other programmed controller and/or a sleep mask having a video display and/or acoustic outputs. The audio media may be stored on a computer memory, a flash drive or other medium.

In further accord with these and other objects, the present invention discloses method providing all-night sleep management comprising determining preselected sleep parameters of a predetermined all-night sleep management paradigm providing all-night sleep therapy selected to provide all-night sleep management and implementing the sleep parameters determined in media that when played on a media player of an all-night sleep management system provides all-night sleep management of the predetermined sleeper's sleep cycle being managed by the media player of the all-night sleep management system. The sleep parameter determining step in different embodiments could be determined in advance to correspond to those of average sleepers or custom-measured for each individual sleeper.

### BRIEF DESCRIPTION OF THE DRAWINGS

These and other objects, inventive aspects and advantageous features of the present invention will become apparent as the invention becomes better understood by referring to the following, solely exemplary, detailed description of the presently preferred embodiments, and to the drawings, wherein:

FIG. 1 is a pictorial diagram of the principles of the system and method providing all-night sleep management in accord with the present invention;

FIG. 2 is a block diagram of the principles of the system providing all-night sleep management in accord with the present invention

FIG. 3 is a block diagram illustrating one embodiment of the system providing all-night sleep management in accord with the present invention;

FIG. 4 is a block diagram illustrating another embodiment of the system providing all-night sleep management in accord with the present invention; and

FIG. 5 is a block diagram illustrating the method providing all-night sleep management in accord with the present invention.

### DETAILED DESCRIPTION OF THE PRESENTLY PREFERRED EMBODIMENTS

Referring now to FIG. 1, generally designated at 10 is a pictorial diagram illustrating the principles of the system and method providing all-night sleep management of the present invention. Circle 12 represents the overall awake-to-sleep-to-wake-up-refreshed cycle of a typical sleeper. As will be readily appreciated, the cycle repeats day-in, day-out, between awake and sleep phases generally designated 14, 16. In accord with the present invention as appears more fully hereinbelow, system and method providing all-night sleep management are disclosed to better integrate the sleep phase 16 into the wake phase 14 with the result that the individual's sense of wholeness and overall well-being is promoted and improved day-in-day-out.

Referring now to FIG. 2, generally designated at 20 is a block diagram of the principles of the system providing all-night sleep management in accord with the present invention. Block 22 represents an all-night sleep management paradigm providing all-night sleep therapy. Any predetermined paradigm 22 determined to provide all-night sleep therapy may be employed. Block 24 represents media implementing the all-night sleep management paradigm 22. Any suitable technique or techniques implementing a predetermined all-night sleep management paradigm 22 in the media 24 may be employed.

The media 24 implementing the all-night sleep management paradigm 22 has a duration that corresponds to the duration of the go-to-sleep-to-wake-up-refreshed-cycle of a predetermined sleeper and predetermined content determined to implement a predetermined all-night sleep management paradigm determined to provide all-night sleep management. The media 24 is played by a media player 26 providing a signal schematically illustrated by wavy arrow 28. The signal 28 interacts with a sleeper, not shown, in room schematically illustrated by dashed box 30 to integrate the sleep cycle of the sleeper whose sleep cycle is being managed by the all-night sleep management system into the wake cycle of the entire wake-sleep cycle, with the result that upon awaking day-in-day-out, the individual's sense of wholeness and overall well-being is promoted and improved.

The media 24 may be audio, video or other media; the signal 28 may be an audio, video or other signal and the media player 26 may be an audio, video and/or other media player. The media's content may be live streamed (for example, from a remote beach or other location), prerecorded or controllably produced by a programmed controller of the media player of the all-night sleep management system of the present invention in response to user input.

Any suitable technique including pacing, sleep enhance effects, for example, as shown and described in U.S. Pat. No. 7,749,155, binaural beats, waveform manipulation, delta, theta etc. brainwave entrainment, light frequency, amplitude and color modulation, voice over coaching to induce sleep, meditation etc., or other techniques known to those skilled in the art may be employed in order to implement the predetermined all-night sleep management paradigm 22 in the media 24 for play on the media player 26.

It will be appreciated the presently preferred and disclosed embodiments of the system providing all-night sleep management in accord with the present invention shown and described herein are exemplary only and many different all-night sleep management paradigms and many different ways of implementing the all-night sleep management paradigms will become apparent to those of skill in the art without departing from the inventive concepts.

Referring now to FIG. 3, generally designated at 40 is a block diagram illustrating one presently preferred embodiment of a system providing all-night sleep management in accord with the present invention. As shown by block 42, the predetermined all-night sleep management paradigm of the system 40 providing all-night sleep management is determined to allow the sleeper whose sleep cycle is being managed by the all-night sleep management system 40 to fall and remain asleep all-night long without disruptions that consciously or unconsciously could disrupt the rest and sleep of sensitive sleepers or prevent falling asleep. The predetermined paradigm 42 determined to allow the sleeper whose sleep cycle is being managed by the all-night sleep management system 40 to fall and remain asleep all-night long without disruptions that consciously or unconsciously could disrupt the rest and sleep of sensitive sleepers is implemented on media 44 for play on a media player 46 that is part of said all-night sleep management system 40. The media 44 is audio media that is of duration that corresponds to the duration of the overall awake-to-sleep-to-wake-up-refreshed sleep cycle of the predetermined sleeper whose sleep cycle is being managed by the all-sleep system and that has prerecorded natural, tinittus or other noise masking, rest promoting or sleep inducing sounds stored thereon having a repeat cycle no less in duration than the duration of the overall awake-to-sleep-to-wake-up-refreshed sleep cycle of the predetermined sleeper's sleep cycle being managed by the media player of the all-night sleep management system 40.

When the audio media 44 is played by an audio player 46, it provides an audio signal schematically illustrated by arrow 48. The audio signal 48 is free from potentially disruptive transitions all night long which helps to allow sensitive sleepers to fall and stay asleep and prevents waking up of otherwise sensitive sleepers the entire sleep cycle of a sleeper, not shown, in room schematically illustrated by dashed box 50, with the result that upon awaking day-in-day-out, the individual's sense of wholeness and overall well-being is promoted and improved.

Referring now to FIG. 4, generally designated at 60 is a block diagram illustrating another embodiment of a system providing all-night sleep management in accord with the present invention. As shown by the block 62, a predetermined all-night sleep management paradigm is determined to include an initial sleep transition phase, a terminal wake-up phase and at least one phase intermediate the initial and terminal phases that together cover the duration of the entire sleep cycle of the overall awake-to-sleep-to-wake-up-refreshed sleep cycle of the predetermined sleeper whose sleep cycle is being managed by the all-night sleep system 60. As shown by block 64, the media implementing the predetermined all-night sleep management paradigm 62 determined to include an initial sleep transition phase, a terminal wake-up phase and at least one phase intermediate the initial and terminal phases is an audio file having a go-to-sleep segment implementing the initial sleep transition phase, a terminal wake-up audio segment implementing the terminal wake-up-phase and at least one audio segment intermediate the initial and terminal go-to-sleep and wake-up segments implementing each at least one intermediate sleep phase of the paradigm 62 that together cover the entire sleep cycle of a predetermined sleeper, not shown, whose sleep cycle is being managed by the all-night sleep management system 60.

Delta frequencies in the one and one tenth to five Hertz frequency range (1.1-5.0 Hz) are known to implement Deep sleep phases; Theta frequencies in the five and one tenth to eight Hertz range (5.1-8.0 Hz) are known to implement REM and dream state sleep phases; Alpha frequencies in the eight and one tenth to twelve Hertz range (8.1-12.0 Hz) are known to implement relaxed awareness (usually but not necessarily as in falling asleep or waking) phases; and Sigma and Beta frequency ranges (12.1-15.0 Hz, 15.1-30.0 Hz) are known to implement full awareness phases.

After falling asleep, in a typical normal sleep pattern an individual will transition from a relaxed state into a deeper sleep state. This deeper sleep state will normally include a series of REM and NREM phases. A typical phase is anywhere between seventy to one hundred and twenty (70-120) minutes in length and will generally consist of the following pattern: REM sleep ten to twenty-five (10 to 25) minutes, NREM (deep) sleep twenty to forty (20 to 40) minutes, and transition back to REM five to ten (10 to 10) minutes. Once these series of sleep cycles are “completed,” then the body will naturally transition slowly from the REM sleep into a wake pattern.

In one presently preferred and exemplary embodiment, for a sleep cycle of time “t,” media 64 is provided having content in seven (VII) segments consisting of (I) an initial go-to-sleep segment of from five (5) to ten (10) minutes of sounds with Alpha wave entrainment; multiple intermediate segments having (II) from ten (10) to twenty (20) minutes of sounds with Theta wave entrainment, (III) from twenty (20) to thirty (30) minutes of sounds with Delta wave entrainment, (IV) from fifteen (15) to thirty (30) minutes of Theta wave entrainment, and (V) from fourteen (14) to thirty-five (35) minutes of Delta wave entrainment; followed by (VI) a repeat of segments (II) thru (V) until, for example, “t-15” minutes; and (VII) a terminal wake up segment of fifteen (15) minutes of sounds with Alpha wave entrainment.

When the audio file 64 is played by an audio player 66, it provides an audio signal 68. When played, the initial go-to-sleep segment of the audio media 64 corresponding to the initial sleep transition phase of the paradigm 62 serves to induce a relaxed state. When the intermediate segments of the audio file 64 corresponding to the intermediate sleep phases of the paradigm 62 are played by the audio player 66, the corresponding signal 68 portions respectively serve to sequentially repetitively induce REM sleep and to induce NREM sleep. When the terminal wake-up segment of the audio file 64 is being played, the corresponding portion of the signal 68 interacts with the sleeper to provide a relaxed wake-up. In this manner, the entire sleep cycle of a sleeper, not shown, in room schematically illustrated by dashed box 70, is managed all-night long by the all-night sleep management system 60 with the result that upon awaking day-in-day-out, the individual's sense of wholeness and overall well-being is promoted and improved.

It will be appreciated by those of skill in the art that different all-night sleep management paradigms having different wake-up and go-to-sleep segments and/or single and/or different intermediate segments could be employed in accord with the present invention without departing from the inventive concepts.

Referring now to FIG. 5, generally designated at 80 is a block diagram illustrating the method providing all-night sleep management in accord with the present invention. As shown by block 82, in a first step, the sleep parameters of a predetermined all-night sleep management paradigm providing all-night sleep therapy of a predetermined sleeper are determined. These parameters may include, for example, duration and sequencing of segments implementing relaxation, REM, NREM and/or wake-up phases, duration of sleep over the sleep phase of the daily sleep/wake cycle, and, among other things, optimal wake-up alarm time. In different embodiments, determination of the sleep parameters is made so as to correspond to those of average sleepers and is custom-made to each individual sleeper. Custom measurements may be done in a commercial or hospital sleep lab or center or by use of home testing or monitoring equipment.

As shown by the block 84, the measured sleep parameters of the all-night sleep management paradigm are implemented in media for play on the media player of an all-night sleep management system. As will be appreciated, depending on the all-night sleep management paradigm implemented, the media may contain one or more segments implementing the corresponding portions of each particular all-night sleep management paradigm. The implementation in media may be done by providing pre-recorded media and/or implemented in media by a programmed controller of the media player of the all-night sleep management system responsive to user total time of sleep selection and a forward set alarm time to coordinate brain wave entrainment of different initial, intermediate and wake-up segments implementing corresponding portions of different all-night sleep-management paradigms that may be preprogrammed in the programmed controller or selectable in whole or in part in response to user input paradigm and/or paradigm phase selection.

As shown by block 86, the media is played on a media player to provide all-night sleep management of the predetermined sleeper determined to either be customized to each individual sleeper or determined for an average sleeper or semi-customized for multiple “average” sleepers.

Many modifications of the presently disclosed invention will become apparent to those of skill in the art without departing from the inventive concepts.

What is claimed is:

1. Apparatus for use in an all-night sleep management system, comprising:
media for play on a media player that is part of said all-night sleep management system that when played on the media player has a duration that corresponds to the duration of the overall awake-to-sleep-to-wake-up-refreshed sleep cycle of a predetermined sleeper whose sleep cycle is being managed by the media player of the all-night sleep management system and has predetermined content stored thereon determined to implement all-night sleep management of a predetermined all-night sleep management paradigm providing all-night sleep therapy of the predetermined sleeper's sleep cycle being managed by the media player of the all-night sleep management system.

2. The invention of claim 1, wherein said predetermined sleeper is an average sleeper and said duration is determined for said average sleeper.

3. The invention of claim 1, wherein said predetermined sleeper is each individual sleeper and said duration is determined for each said individual sleeper.

4. The invention of claim 1, wherein the predetermined all-night sleep management paradigm providing all-night sleep therapy is determined to allow the sleeper whose sleep cycle is being managed by the all-night sleep management system to fall and remain sleep all-night long without disruptions that consciously or unconsciously could disrupt the rest and sleep of sensitive sleepers; wherein the media of duration that corresponds to the duration of the overall awake-to-sleep-to-wake-up-refreshed sleep cycle implementing said paradigm is audio media having prerecorded at least one noise-masking, rest-promoting and sleep-inducing sounds stored thereon having a repeat cycle; and wherein said repeat cycle of said at least one sound of said audio media is no less in duration than the duration of the overall awake-to-sleep-to-wake-up-refreshed sleep cycle of the predetermined sleeper's sleep cycle being managed by the media player of the all-night sleep management system.

5. The invention of claim 4, wherein said predetermined audio content includes tinittus masking sounds.

6. The invention of claim 4, wherein said predetermined audio content includes natural sounds.

7. The invention of claim 4, wherein said predetermined audio content includes sleep enhance effects.

8. The invention of claim 4, wherein said predetermined audio content includes voice over effects.

9. The invention of claim 4, wherein said predetermined audio content includes brainwave entrainment.

10. The invention of claim 1, wherein said predetermined audio content includes live streaming of sounds from a remote location.

11. The invention of claim 1, wherein the predetermined all-night sleep management paradigm is determined to include an initial sleep transition phase, a terminal wake-up phase and at least one phase intermediate the initial and terminal phases that together cover the duration of the entire sleep cycle of the overall awake-to-sleep-to-wake-up-refreshed sleep cycle of the predetermined sleeper whose sleep cycle is being managed by the all-night sleep management system; wherein the media implementing the predetermined all-night sleep management paradigm determined to include an initial sleep transition phase, a terminal wake-up phase and at least one phase intermediate the initial and terminal phases has a go-to-sleep segment implementing the initial sleep transition phase, a terminal wake-up segment implementing the terminal wake-up-phase and at least one segment intermediate the initial and terminal go-to-sleep and wake-up segments implementing the at least one intermediate sleep phases that together cover the entire sleep cycle of a predetermined sleeper whose sleep cycle is being managed by the all-night sleep management system.

12. The invention of claim 11, wherein said predetermined sleeper is an average sleeper and said duration is determined for said average sleeper.

13. The invention of claim 11, wherein said predetermined sleeper is each individual sleeper and said duration is determined for each said individual sleeper.

14. The invention of claim 11, wherein the media is audio media and the media player is an audio player.

15. The invention of claim 14, wherein the audio media includes initial, terminal and intermediate brainwave entrainment segments.

16. A method providing all-night sleep management comprising the steps of:
determining preselected sleep parameters of a predetermined all-night sleep management paradigm determined to provide all-night sleep therapy selected to provide all-night sleep management; and implementing the sleep parameters determined in media that when played on the media player of an all-night sleep management system provides said all-night sleep management.

17. The method of claim 16, wherein said determining step is determined in relation to the average sleeper.

18. The method of claim 16, wherein said determining step is custom determined in relation to each individual sleeper.

19. The method of claim 16, wherein the media player includes a programmed controller, wherein said programmed controller is operative in response to user input to determine said preselected sleep parameters of said predetermined sleep management paradigm and to implement the sleep parameters determined in media that when played on the media player provides all-night sleep management.

20. The method of claim 19, wherein said user input includes duration.

21. The method of claim 19, wherein said predetermined paradigm includes initial, terminal and at least one intermediate phases.

22. The method of claim 16, wherein said implementing step implements the parameters determined in prerecorded media.
